(19) United States
(12) Patent Application Publication
(10) Pub. No.: US 2009/0310906 A1
(43) Pub. Date: Dec. 17, 2009

(54) Optical device and producing method thereof

(75) Inventor: Tetsuya Miyatake, Kawasaki (JP)

Correspondence Address: STAAS & HALSEY LLP, SUITE 700, 1201 NEW YORK AVENUE, N.W., WASHINGTON, DC, 20005, US

(73) Assignee: FUJITSU LIMITED, Kawasaki (JP)

(21) Appl. No.: 12/385,006

(22) Filed: Mar. 27, 2009

(30) Foreign Application Priority Data
Jun. 17, 2008 (JP) 2008-158090

(51) Int. Cl. G02B 6/12; A61N 5/00

(52) U.S. Cl. 385/14; 250/492.1

(57) ABSTRACT

An optical device includes: a crystal substrate; an optical waveguide formed in the crystal substrate; and an amorphous area formed in the crystal substrate, adjacent to a curved waveguide of the optical waveguide. The amorphous area is formed by irradiation of an ultrashort pulse laser.

### CROSS-REFERENCE TO RELATED APPLICATION

This application is based upon and claims the benefit of priority of the prior Japanese Patent Application No. 2008-158090, filed on Jun. 17, 2008, the entire contents of which are incorporated herein by reference.

### FIELD

The present invention relates to an optical device having an optical waveguide integrated on a substrate.

### BACKGROUND

In optical communication technology, the demand for miniaturization of transmission systems has been increasing more and more recently. In order to respond to this demand, it is necessary to integrate functions which have heretofore been realized by connecting a plurality of individual optical devices on one substrate (one chip), and to further miniaturize the size of each optical device. As an important technique for such miniaturization and integration, adoption of a curved waveguide for the optical waveguide in the optical device can be mentioned. Recently as the curved waveguide, a three-dimensional curved waveguide (that is to say, one that is also bent in the depth direction of the device) is being studied.

When designing a curved waveguide in a substrate, it is obvious that reduction of the radius of curvature as much as possible is effective for miniaturization. On the other hand however, as the radius of curvature decreases, the optical loss (radiation loss) increases, leading to performance deterioration of the optical device having the optical waveguide, and hence care may be taken. Techniques for reducing such an optical loss are disclosed in Japanese Laid-Open Patent Publication No. 2007-094440 (reference document 1), Japanese Patent Publication No. 2847844 (reference document 2), and Japanese Patent Publication No. 2855676 (reference document 3).

Reference document 1 discloses a technique for arranging an air layer on a side of the waveguide by making a curved waveguide into a ridge structure on the substrate, and using the air layer as a cladding. Moreover reference documents 2 and 3 disclose a technique for forming a cladding area for the optical waveguide in a substrate surface layer on a side of the waveguide by using external diffusion or ion exchange.

All of the techniques disclosed in reference documents 1, 2 and 3 have a point to be solved in that they may not be applied to a three-dimensional curved waveguide passing through the inside of the substrate away from the substrate surface. That is to say, the curved waveguide buried inside the substrate may not have the ridge structure in reference document 1, and the cladding area may not be formed inside the substrate away from the substrate surface, by the external diffusion or ion exchange used in reference documents 2 and 3.

Focusing on the above point, it is required that an optical device structure and a producing method thereof that, even for an optical waveguide formed inside a substrate, is capable of providing a cladding for the curved waveguide.

### SUMMARY

An optical device proposed herein includes: a crystal substrate; an optical waveguide formed in the crystal substrate; and an amorphous area formed in the crystal substrate, adjacent to at least a part of the optical waveguide.

Moreover as a method for producing the optical device, there is proposed herein a method including a process of irradiating an ultrashort pulse laser onto a portion adjacent to an optical waveguide in a crystal substrate in which the optical waveguide is formed, to form an amorphous area in the adjacent portion.

Additional objects and advantage of the invention will be set forth in part in the description which follows, and in part will be obvious from the description, or may be learned by practice of the invention. The object and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

FIG. 1A is a plan view of an optical device according to a first embodiment;

FIG. 1B is a sectional view of the optical device according to the first embodiment;

FIG. 2 is a plan view of an optical device according to a second embodiment;

FIG. 3A is a plan view of an optical device according to a third embodiment;

FIG. 3B is a sectional view of the optical device according to the third embodiment;

FIG. 4A is a plan view of an optical device according to a fourth embodiment;

FIG. 4B is a side view of the optical device according to the fourth embodiment;

FIG. 4C is a sectional view of the optical device according to the fourth embodiment;

FIG. 5 is an explanatory diagram of an amorphous area forming method;

FIG. 6 is an explanatory diagram concerning irradiation power of a femtosecond laser used for amorphization; and

FIG. 7 is a diagram illustrating results of optical waveguiding simulation by a beam propagation method.

### DESCRIPTION OF EMBODIMENTS

The optical device according to the present proposal is provided with an amorphous area adjacent to an optical waveguide. It is known that when an amorphized part is formed in a crystal substrate, a refractive index of the amorphized part decreases more than for the other parts. On the other hand, the optical waveguide in the crystal substrate is a part with a refractive index higher than for the other parts, and hence, if the amorphous area is formed adjacent to the optical waveguide, a difference in the refractive index between these parts increases. That is to say, the amorphous area can be used as the cladding of the optical waveguide.

Such an amorphous area can be formed inside a crystal substrate by irradiation of an ultrashort pulse laser. Therefore even when an optical waveguide is formed inside the substrate away from the substrate surface, the amorphous area can be formed along the optical waveguide inside the substrate by adjusting a focal position of the laser. That is to say, the amorphous area can be formed in a required position even inside the substrate away from the substrate surface. Therefore even for a three-dimensional curved waveguide, a structure of an optical device capable of having a cladding, and a producing method thereof can be provided.

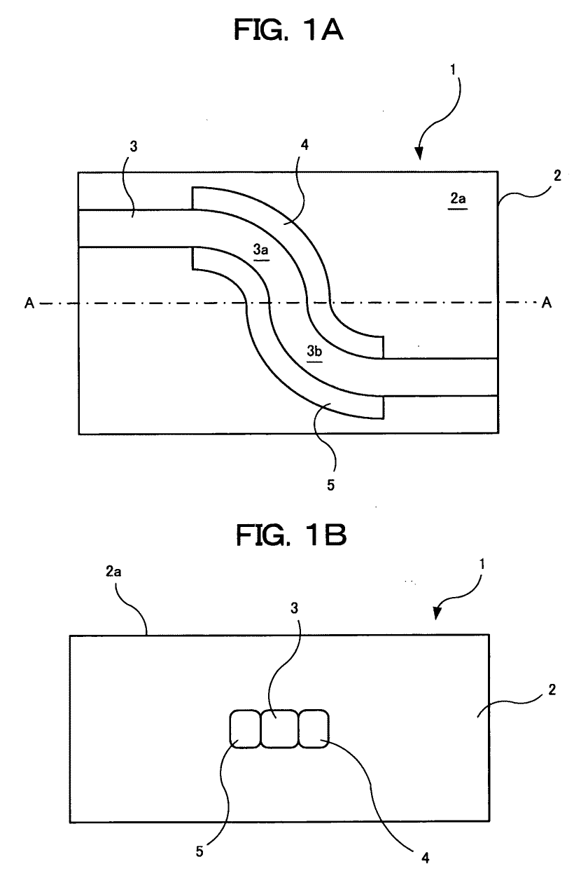

FIG. 1A is a plan view of an optical device according to a first embodiment, and FIG. 1B illustrates a sectional view on the line A-A thereof.

An optical device 1 according to the first embodiment employs a lithium niobate (LiNbO₃) substrate (hereinafter, referred to as an LN substrate) 2 as the crystal substrate, and an optical waveguide 3 is buried and formed inside the substrate away from a substrate surface 2a. The optical waveguide 3 is formed in an S-shaped curved planar shape having curved waveguides 3a and 3b at two places along the way.

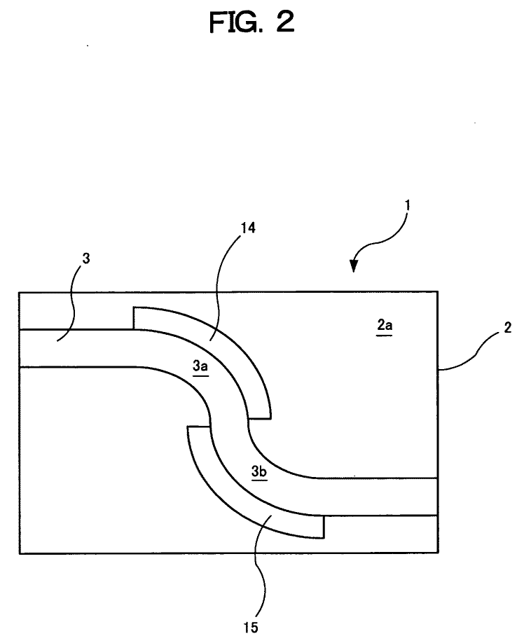

For such an optical waveguide 3, in the first embodiment, amorphous areas 4 and 5 are formed adjacent to both of an internal circumference side and an external circumference side of the curved waveguides 3a and 3b, so as to sandwich the curved waveguides 3a and 3b therebetween. That is to say, one amorphous area 4 is continuously formed from the external circumference side of the curved waveguide 3a toward the internal circumference side of the curved waveguide 3b, and the other amorphous area 5 is continuously formed from the internal circumference side of the curved waveguide 3a toward the external circumference side of the curved waveguide 3b. However, if the cladding for the curved waveguides 3a and 3b is at least on the external circumference side of the curve, a necessary minimum function can be obtained. Therefore, as illustrated in FIG. 2, only amorphous areas 14 and 15 adjacent to the external circumference side of the curved waveguides 3a and 3b may be formed.

If the optical waveguide 3 in the LN substrate 2 is one that is formed in the substrate surface layer, it can be formed by thermal diffusion of titanium (Ti) as illustrated in the aforementioned reference document 1. However in the case where this is one that is formed inside the substrate as in the first embodiment, it can be formed by irradiation of an ultrashort pulse laser. For example, a technique for forming an optical waveguide by irradiating a femtosecond laser onto the crystal substrate to induce a physical change at a focal position thereof, and increase the refractive index at the portion where the physical change is induced, is disclosed in the following papers 1, 2, 3 and 4. Such an optical waveguide 3 formed by irradiation of a femtosecond laser is a part where the refractive index is higher than in other parts of the LN substrate 2.
[Paper 1] “Femtosecond Laser Inscription of Low Insertion Loss Waveguides in Z-Cut Lithium Niobate”, Henry T. Bookey et al., IEEE Photon. Technol. Lett. 19 (12), 892-489 (2007)
[Paper 2] “Efficient frequency doubling in femtosecond laser-written waveguides in lithium niobate”, Jonas Burghoff et al., Appl. Phys. Lett. 89, 081108 (2006)
[Paper 3] “Optical waveguide fabrication in z-cut lithium niobate (LiNbO3) using femtosecond pulses in the low repetition rate regime”, R. R. Thomson et al., Appl. Phys. Lett. 88, 111109 (2006)
[Paper 4] “Microstructure in Lithium Niobate by Use of Focused Femtosecond Laser Pulses”, Li Gui et al., IEEE Photon. Technol. Lett. 16(5), 1337-1339 (2004)

On the other hand, for example in the case of lithium niobate, a situation where the refractive index is decreased by about 0.1 as compared to when in a crystalline state due to amorphization, is reported in the following Paper 5.
[Paper 5] “RF Sputtering of LiNbO3 Thin Films”, G. H. Hewig et al., Thin Solid Films 88, 67-74 (1982)

Consequently, for example in the LN substrate 2 having a refractive index of 2.2, the refractive index of the amorphous areas 4, 5, 14, and 15 becomes 2.1 (=2.2-0.1), being lower than the refractive index of the LN substrate 2 by 4.5%, so that the refractive index difference with the optical waveguide 3 is increased. That is to say, the amorphous areas 4, 5, 14, and 15 function as the cladding, so that the optical loss in the curved waveguides 3a and 3b can be reduced. As a result, the radius of curvature of the curved waveguide can be reduced further than in a conventional case, contributing to miniaturization of the optical device.

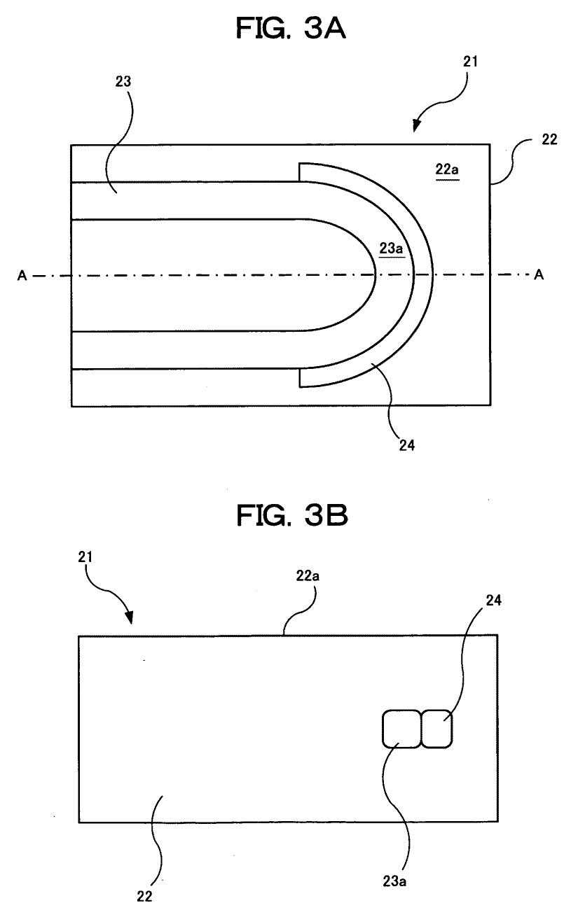

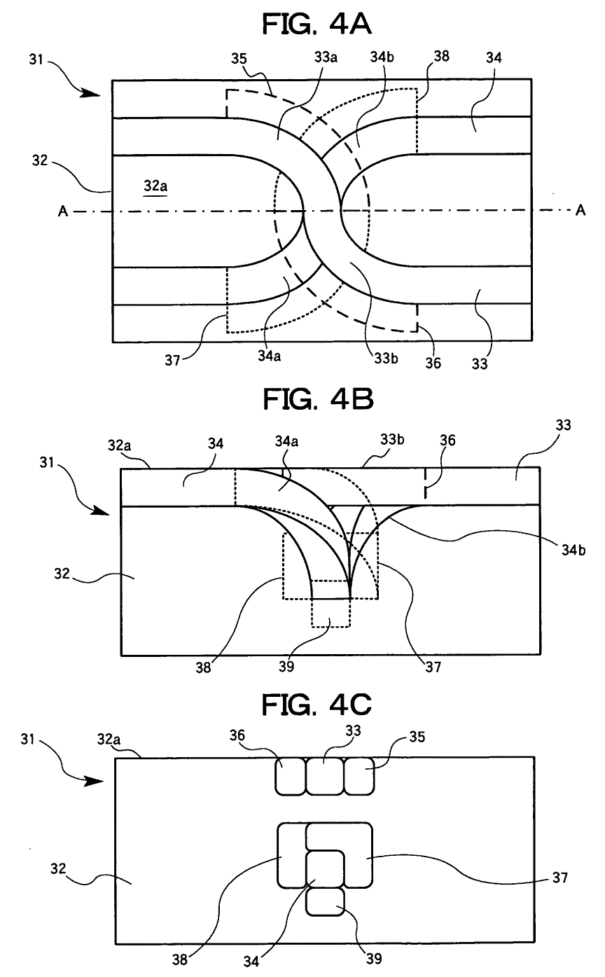

FIG. 3 and FIG. 4 illustrate third and fourth embodiments of an optical device, as other examples.

An optical device 21 according to the third embodiment in FIG. 3 is provided with an optical waveguide 23 in a folded-back U-shaped planar shape on an end face the same as the light incident end face, in an LN substrate 22 serving as the crystal substrate. An amorphous area 24 is formed for a portion of a curved waveguide 23a in the optical waveguide 23, adjacent to the external circumference side thereof. In this way, the optical waveguide 23 can be folded back with the amorphous area 24 as the cladding. Therefore the length of the optical device 21 can be shortened enabling miniaturization. The optical waveguide 23 is formed buried inside the substrate away from a substrate surface 22a, similarly to the first and second embodiments, and can be formed for example, by irradiation of the femtosecond laser as mentioned above. FIG. 3A is a plan view and FIG. 3B is a sectional view on the line A-A thereof.

An optical device 31 according to the fourth embodiment in FIG. 4 is formed by crossing two optical waveguides 33 and 34 having an S-shaped curved planar shape, in an LN substrate 32 serving as the crystal substrate. One optical waveguide 33 is formed along a substrate surface 32a, and the other optical waveguide 34 bends to the inside of the substrate in an arched shape away from the substrate surface 32a at an intersection, so as to avoid the one optical waveguide 33. FIG. 4A is a plan view, FIG. 4B is a side view, and FIG. 4C is a sectional view on the line A-A.

The one optical waveguide 33 is the same as that in the second embodiment, in which amorphous areas 35 and 36 are formed as the cladding, adjacent to the external circumference side of two curved waveguides 33a and 33b. Such an optical waveguide 33 can be formed by thermal diffusion of titanium, or by femtosecond laser irradiation as described above.

The other optical waveguide 34 has three-dimensional curved waveguides 34a and 34b which curve and bend into the inside of the substrate for crossing. That is to say, the curved waveguides 34a and 34b curve in a horizontal direction along the LN substrate 32, and also curve in a perpendicular direction (thickness direction) to the LN substrate 32. Such an optical waveguide 34 is preferably formed by the above-mentioned femtosecond laser irradiation. Amorphous areas 37, 38, and 39 are formed for the curved waveguides 34a and 34b, adjacent to horizontal and perpendicular external circumferences. The amorphous areas 37 and 38 are formed in portions above and on the side, which become the external circumferential sides, of the curved waveguides 34a and 34b where these start to bent into the inside of the substrate. Moreover the amorphous area 39 is formed below the arch shape portion, which is the external circumferential side thereof, the arch shape portion being arranged to spanning under the one optical waveguide 33 along the curved waveguides 34a and 34b.

The optical devices according to the above respective embodiments can be used for example as a Mach-Zehnder optical modulator, or an optical switch.

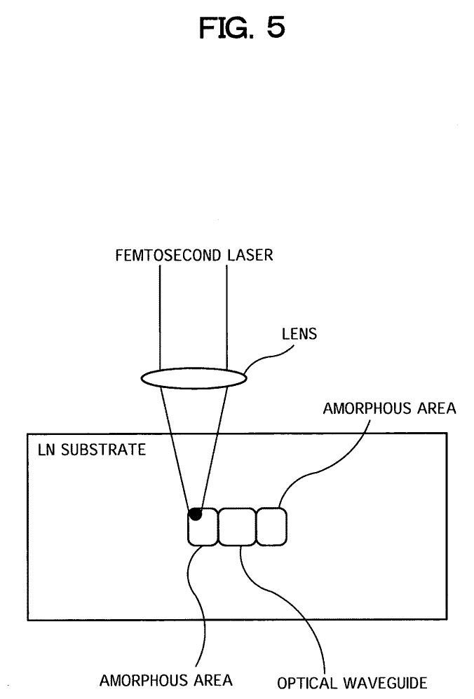

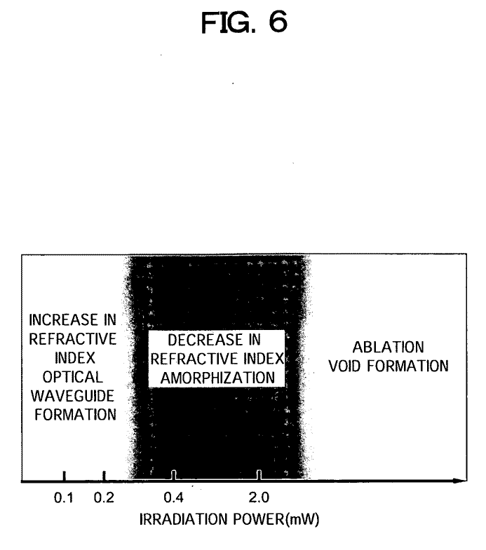

The amorphous area in the above respective embodiments can be formed by irradiation of an ultrashort pulse laser, for example, a femtosecond laser. That is to say, as shown in FIG. 5, the amorphous area can be formed by passing a femtosecond laser through a lens to adjust the focal position, and irradiating this onto a portion adjacent to the optical waveguide inside the LN substrate. The femtosecond laser is continuously moved so as to scan the area to be amorphized. FIG. 6 is an explanatory diagram of the irradiation power at this time.

As described above, the femtosecond laser is also used for forming the optical waveguide. However, the irradiation power in this case is within a range from about 0.1 to 0.2 mW and works so as to increase the refractive index of the LN substrate. On the other hand, a technique for forming a void (depletion) along a light propagating layer by the femtosecond laser is disclosed in Japanese Laid-Open Patent Publication No. 2004-029286. This is for inducing ablation in the LN substrate as the physical change, and the irradiation power of the femtosecond laser in this case takes a value exceeding 2 mW. In contrast to these cases, when the irradiation power of the femtosecond laser is within a range from 0.4 to 2.0 mW, amorphization is induced in the LN substrate, to decrease the refractive index of the LN substrate, however ablation is not caused. In other words, when the irradiation power of the femtosecond laser irradiated onto the LN substrate is increased, the physical properties of the LN substrate change in the order of: an increase in refractive index; then amorphization (a decrease in refractive index); and then ablation. By using the power range of amorphization within these power ranges, the amorphous area can be formed by irradiation of the femtosecond laser. If ablation is caused in the LN substrate, there is concern that scattering of light might increase due to the void. Therefore, the amorphous area may be used.

As a specific example, in the LN substrate, an irradiation condition of the femtosecond laser for forming the amorphous area at a position of 40 to 70 μm depth from the substrate surface is such that the pulse width is 80 to 100 femtoseconds, the irradiation power is 0.5 to 2.0 mW, and the pulse repetition frequency is 100 to 1000 Hz.

In this way, the amorphous area in the LN substrate can be formed by irradiation of the femtosecond laser. Therefore the amorphous area can be formed in an adjacent portion even for a three-dimensional curved waveguide, thereby contributing to miniaturization of the optical device by adopting the three-dimensional curved waveguide. Besides, the LN substrate has been explained specifically, however, the present invention can be applied similarly to a crystal which is amorphized by laser irradiation to decrease the refractive index of the amorphous area.

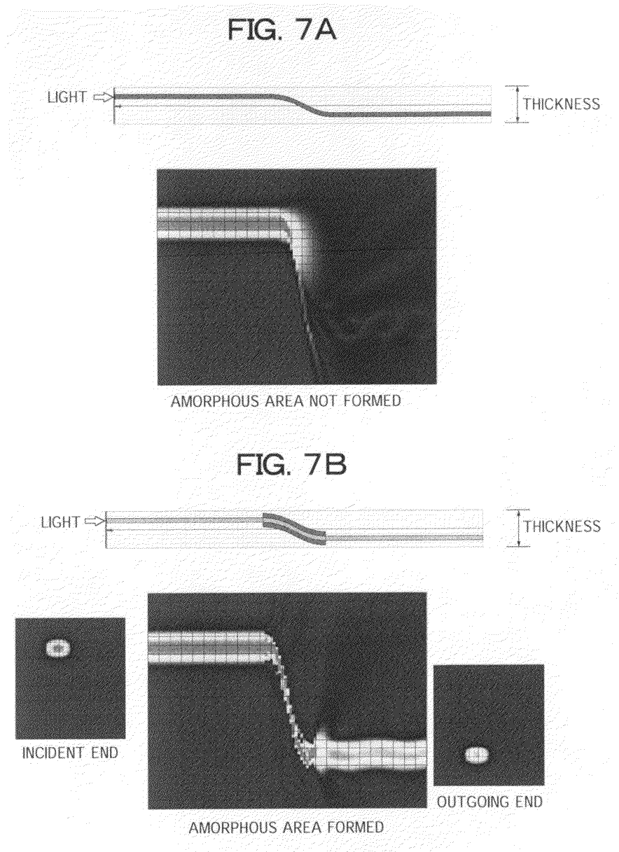

The results of optical waveguiding simulation obtained by a beam propagation method (BPM) are illustrated in FIG. 7. FIG. 7A illustrates an optical waveguide in which the amorphous area is not formed adjacent thereto and FIG. 7B illustrates an optical waveguide in which the amorphous area is formed adjacent thereto. Both of the optical waveguides are formed in the LN substrate, and have a curved waveguide curved in an S-shape toward the thickness direction of the substrate (from top to bottom in the figures) along the optical waveguide. The optical waveguide in FIG. 7B has the amorphous area formed adjacent to the curved waveguide, above and below the curved waveguide. Calculation is performed assuming that; the radius of curvature of the curved waveguide is 0.5 mm, the wavelength of the incident light is 1550 nm, and the light propagates from left to right in the figures.

As illustrated in the figures, in the optical waveguide in FIG. 7A having no amorphous area, the incident light scatters when it approaches the curved waveguide, and does not reach an outgoing end. On the other hand, in the optical waveguide in FIG. 7B having the amorphous area, scattering at the curved waveguide is suppressed, and the light reaches the outgoing end. Thus, by forming the amorphous area adjacent to the optical waveguide, it is possible to suppress the optical loss in the curved waveguide.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiments of the present invention have been described in detail, it should be understood that the various changes, substitutions, and alterations could be made hereto without departing from the spirit and scope of the invention.

What is claimed is:

1. An optical device including:
a crystal substrate;
an optical waveguide formed in the crystal substrate; and
an amorphous area formed in the crystal substrate, adjacent to at least a part of the optical waveguide.

2. An optical device according to claim 1, wherein
the optical waveguide has at least a partially curved waveguide, and
the amorphous area is formed adjacent to an outer circumference side of the curved waveguide.

3. An optical device according to claim 1, wherein
the crystal substrate is a lithium niobate (LiNbO₃) substrate.

4. An optical modulator including an optical device, the optical device comprising:
a crystal substrate;
an optical waveguide formed in the crystal substrate; and
an amorphous area formed in the crystal substrate, adjacent to at least a part of the optical waveguide.

5. An optical modulator according to claim 4, wherein
the optical waveguide of the optical device has at least a partially curved waveguide, and
the amorphous area of the optical device is formed adjacent to an outer circumference side of the curved waveguide.

6. An optical modulator according to claim 4, wherein
the crystal substrate of the optical device is a lithium niobate (LiNbO₃) substrate.

7. An optical switch including an optical device, the optical device comprising:
a crystal substrate;
an optical waveguide formed in the crystal substrate; and
an amorphous area formed in the crystal substrate, adjacent to at least a part of the optical waveguide.

8. An optical switch according to claim 7, wherein
the optical waveguide of the optical device has at least a partially curved waveguide, and
the amorphous area of the optical device is formed adjacent to an outer circumference side of the curved waveguide.

9. An optical switch according to claim 7, wherein
the crystal substrate of the optical device is a lithium niobate (LiNbO₃) substrate.

10. A method for producing an optical device, comprising
irradiating an ultrashort pulse laser onto a portion adjacent to an optical waveguide in a crystal substrate in which the optical waveguide is formed, to form an amorphous area in the adjacent portion.

11. A method for producing an optical device according to claim 10, wherein
an irradiation power of the ultrashort pulse laser is greater than a power to induce an increase in refractive index of the crystal of the crystal substrate, and less than a power to produce ablation of the crystal of the crystal substrate.

12. A method for producing an optical device according to claim 11, wherein
the crystal substrate is a lithium niobate (LiNbO₃) substrate, and
the ultrashort pulse laser is a femtosecond laser.

13. A method for producing an optical device according to claim 12, wherein
an irradiation power of the femtosecond laser is 0.5 mW to 2.0 mW.

14. A method for producing an optical device according to claim 11, further comprising:
irradiating the ultrashort pulse laser at an irradiation power to induce an increase in refractive index of the crystal of the crystal substrate, to form the optical waveguide.
